(19) United States
(12) Patent Application Publication
(10) Pub. No.: US 2017/0100143 A1
(43) Pub. Date: Apr. 13, 2017

(54) MULTIPLE BARREL CLOT REMOVAL DEVICES

(71) Applicants: STRYKER CORPORATION, Kalamazoo, MI (US); STRYKER EUROPEAN HOLDINGS I, LLC, Kalamazoo, MI (US)

(72) Inventor: Ryan M. Grandfield, Livermore, CA (US)

(21) Appl. No.: 15/285,432

(22) Filed: Oct. 4, 2016

Related U.S. Application Data
(60) Provisional application No. 62/252,811, filed on Nov. 9, 2015.
(60) Provisional application No. 62/238,366, filed on Oct. 7, 2015.

(51) Int. Cl. A61B 17/221

(52) U.S. Cl. CPC A61B 17/221; A61B 2017/2215

(57) ABSTRACT

A clot removal device comprising a clot engaging structure comprising a plurality of interconnected struts forming an open cell pattern, the clot engaging structure having a radially constrained configuration and a radially expanded configuration, wherein when the clot engaging structure is in the radially expanded configuration, a first portion of the clot engaging structure is rolled about itself in a clockwise direction to form a first barrel, and a second portion of the clot engaging structure is rolled about itself in a counter-clockwise direction to form a second barrel that extends in a side-by-side configuration with the first barrel.

### RELATED APPLICATION DATA

The present application claims the benefit under 35 U.S.C. §119 to U.S. Provisional Application Ser. Nos. 62/238,366, filed Oct. 7, 2015 and 62/252,811, filed Nov. 9, 2015. The foregoing applications are hereby incorporated by reference into the present application in its entirety.

### FIELD OF INVENTION

The disclosed inventions are directed to medical devices used to treat and remove obstructions from a blood vessel, such as a blood clot, and more particularly, to embodiments of a multiple barrel clot removal device configured for removing obstructions from a blood vessel.

### BACKGROUND

Various surgical devices have been developed for treating and removing vasculature obstructions (also referred to as clots). Vasculature obstructions include clots such as blood clots in the cerebral vasculature which cause embolic strokes and obstructions in various other locations of the vasculature system which can cause various medical conditions such as venous thrombosis or heart attacks. Vasculature obstructions may form in blood vessels by various mechanisms. For instance, emboli may form at a location in the vasculature and become dislodged and then become lodged in a different blood vessel location. For example, emboli occasionally form around the valves of the heart and then are dislodged and follow the blood flow into the distal regions of the body. Such emboli are particularly dangerous should they migrate to the brain neuro vasculature, and cause an embolic stroke.

In general, surgical devices for treating and removing vasculature obstructions are usually delivered through an intravascular catheter. The surgical devices may treat the blood clot in several ways. In one way, the surgical device may be configured and used to open a clear passageway adjacent a thrombus to allow both blood and medication to bypass the clot. Other devices may be configured to pierce and/or remove a thrombus. These thrombi are often found in tortuous vasculature.

Various surgical devices to treat vascular obstructions such as emboli have been previously developed. The use of inflatable balloons to remove emboli has been practiced for many years. For instance, the “Fogarty catheter” has been used, typically in the periphery, to remove clots from arteries found in legs and in arms. These well known devices have been described in some detail in U.S. Pat. No. 3,435,826, to Fogarty and in U.S. Pat. Nos. 4,403,612 and 3,367,101. These patents describe a balloon catheter in which a balloon material is longitudinally stretched when deflated. In procedures for removing emboli using the Fogarty catheter or other similar catheters, it is typical, first, to locate the clot using fluoroscopy. The embolectomy catheter is then inserted and directed to the clot. The distal tip of the balloon catheter is then carefully moved through the center of the clot. Once the balloon has passed through the distal side of the clot, the balloon is inflated. The balloon catheter is then gradually and gently withdrawn. The balloon acts to pull the clot ahead of the balloon.

Removal of emboli using balloon catheters is rife with potential problems. One such problem occurs during removal of a clot. The resistance to such removal often causes the balloon portion of the catheter to evert over the tip of the catheter. Should the user need to partially deflate the balloon during such a deflation, the distal tip of the balloon may become distended and angulate. Another difficulty with balloon catheters is the possibility of damage to the intima of arteries. Inflation pressures can create forces significant enough to score the vessel lining or dislodge plaque lodged on the vessel wall. In the worst case, the balloon may rupture leaving balloon portions in the bloodstream.

Another surgical device for removing clots is described in U.S. Pat. No. 8,852,205, to Brady et al. In one described embodiment, the surgical device includes a dual tube configuration, in which two tubular structures are connected to each other, each tube having a plurality of struts reinforcing its surface. The tubes are connected to a shaft by respective connecting arms. Brady et al.

describes that the ends of the tubes may be open or closed by inwardly facing struts. However, the tubes create a very rigid structure which cannot be easily compressed for placement into an intravascular insertion catheter, since the tubes independently resist compression.

### SUMMARY

Exemplary embodiments of the disclosed inventions are directed to a clot removal device that comprises a clot engaging structure, the clot engaging structure comprising a plurality of interconnected struts forming an open cell pattern and having a radially constrained configuration and a radially expanded configuration. When the clot engaging structure is in the radially expanded configuration, a first portion of the clot engaging structure is rolled about itself in a clockwise direction to form a first barrel, and a second portion of the clot engaging structure is rolled about itself in a counter-clockwise direction to form a second barrel that extends in a side-by-side configuration with the first barrel.

In some embodiments, the first portion of the clot removal structure terminates along a first edge that does not overlap with a remainder of the first barrel when the clot engaging structure is in the radially expanded configuration, and the second portion of the clot removal structure terminates along a second edge that does not overlap with a remainder of the second barrel when the clot engaging structure is in the radially expanded configuration.

In some embodiments, the first portion of the clot removal structure terminates along a first edge that overlaps with at least a portion of the first barrel when the clot engaging structure is in the radially expanded configuration, and the second portion of the clot removal structure terminates along a second edge that overlaps with at least a portion of the second barrel when the clot engaging structure is in the radially expanded configuration.

In some embodiments, when the clot engaging structure is in the radially expanded configuration, the first barrel has a first barrel diameter, and the second barrel has a second barrel diameter that is smaller than the first barrel diameter.

In some embodiments, the clot engaging structure may further comprise an intermediate portion disposed between the first and second portions, wherein the intermediate portion may comprise a loop and/or a third barrel when the clot engaging structure is in the radially expanded configuration.

In various embodiments, the clot engaging structure is preferably biased to expand, or is otherwise expandable from, the radially constrained configuration to the radially expanded configuration when deployed from a delivery catheter into a blood vessel.

In various embodiments, when the clot engaging structure is delivered to a targeted vascular site proximate a vascular obstruction, and moved or allowed to move from the radially constrained configuration to the radially expanded configuration, the first and second barrels move, be moved, or be allowed to move, respectively, from a radially constrained configuration to a radially expanded configuration to thereby ensnare or encapsulate the vascular obstruction or portions thereof between or within the first and second barrels.

Other and further aspects and features of embodiments of the disclosed inventions will become apparent from the ensuing detailed description in view of the accompanying figures.

### BRIEF DESCRIPTION OF THE DRAWINGS

FIG. 1 is a front perspective view of a multiple barrel clot removal device, constructed according to one embodiment of the disclosed inventions.

FIG. 2 is a cross-sectional view of the clot removal device taken along line 3-3 of FIG. 1.

FIG. 3 is a top view of a clot removal structure that may be used for forming the clot removal device of FIG. 1.

FIG. 4 is a cross-sectional view of an alternative multiple barrel clot removal device, constructed according to another embodiment of the disclosed inventions.

FIG. 5 is a cross-sectional view of still another multiple barrel clot removal device, constructed according still another embodiment of the disclosed inventions.

FIG. 6 is a cross-sectional view of yet another multiple barrel clot removal device, constructed according to yet another embodiment of the disclosed inventions.

FIG. 7 is a cross-sectional view of still another multiple barrel clot removal device, constructed according to still another embodiment of the disclosed inventions.

FIG. 8 is a cross-sectional view of yet another multiple barrel clot removal device, constructed according to still another embodiment of the disclosed inventions.

FIG. 9 is a cross-sectional view of still another multiple barrel clot removal device, constructed according to yet another embodiment of the disclosed inventions.

FIG. 10 is a cross-sectional view of still another multiple barrel clot removal device, constructed according to yet another embodiment of the disclosed inventions.

FIGS. 11A-C are cross-sectional views of the clot removal device constructed according to the embodiments of the disclosed inventions, depicting capturing an embolic obstruction or clot within a blood vessel.

FIG. 12 is a cross-sectional view of still another multiple barrel clot removal device, constructed according to still another embodiment of the disclosed inventions.

FIG. 13 is a cross-sectional view of still another multiple barrel clot removal device, constructed according to still another embodiment of the disclosed inventions.

FIG. 14 is a cross-sectional view of still another multiple barrel clot removal device, constructed according to still another embodiment of the disclosed inventions.

FIG. 15 is a cross-sectional view of still another multiple barrel clot removal device, constructed according to still another embodiment of the disclosed inventions.

### DETAILED DESCRIPTION

Various embodiments of the disclosed inventions are described hereinafter with reference to the figures. The figures are not necessarily drawn to scale, the relative scale of select elements may have been exaggerated for clarity, and elements of similar structures or functions are represented by like reference numerals throughout the figures. It should also be understood that the figures are only intended to facilitate the description of the embodiments, and are not intended as an exhaustive description of the invention or as a limitation on the scope of the invention, which is defined only by the appended claims and their equivalents. In addition, an illustrated embodiment of the disclosed inventions needs not have all the aspects or advantages shown. An aspect or an advantage described in conjunction with a particular embodiment of the disclosed inventions is not necessarily limited to that embodiment and can be practiced in any other embodiments even if not so illustrated.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure. The recitation of numerical ranges by endpoints includes all numbers within that range (e.g., 1 to 5 includes 1, 1.5, 2, 2.75, 3, 3.80, 4, and 5). As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The disclosed embodiments herein directed to clot removal devices having multiple barrels. The disclosed clot removal devices, also referred to as stentrievers, are used for removing obstructions (i.e., blood clots) from a blood vessel.

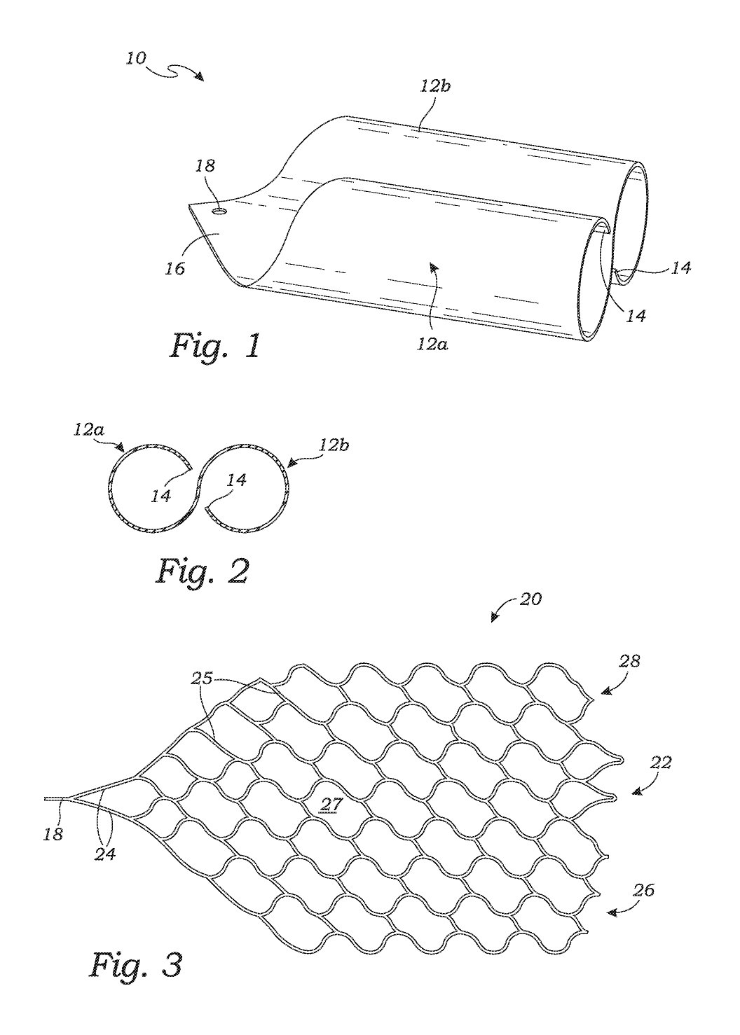

Referring to FIGS. 1 and 2, in one embodiment, the clot removal device 10 comprises a plurality of elongate barrels 12. Each barrel 12 has a length along a longitudinal axis of each barrel in the direction of the elongate length of each barrel 12. The barrels 12 are arranged substantially side by side and substantially parallel to each other along their length. The barrels 12 are connected to each other. As shown in the embodiment of FIGS. 1 and 2, the barrels 12 may be connected to each other along the length of the outside surface of each barrel 12. Alternatively, the barrels 12 may be connected to each other at only discrete locations along their length or by connecting each barrel 12 to a common support structure, such as a frame or body.

Each barrel 12 has a free, unconnected edge 14 extending along substantially the entire length of the barrel 12. Accordingly, the cross-section of each barrel 12 is an open shape, i.e. there is an unconnected edge 14. In the embodiment of FIGS. 1 and 2, the free, unconnected edge 14 terminates short of the other side of the barrel (e.g., forming a partial cylinder). As described below for other embodiments, the edge 14 may overlap the other side (e.g., in a slightly helical shape). Still, in a relaxed state of the device 10 (i.e., with no outside forces bearing on the barrel), the edge 14 may be spatially separated (i.e. not in contact, not overlapping), as shown in FIG. 6, or may be spatially together (i.e., in contact, overlapping) as shown in FIG. 5, from any other structure of the clot removal device, as long as the edge 14 is free and unconnected (i.e., not fixedly attached) from any other structure of the clot removal device.

The elongate barrels 12 may have any suitable cross-sectional shape, such as the circular cross-section shown in the embodiment of FIGS. 1 and 2, resulting in each of the barrels 12 being a partial cylinder. The shape of the barrels 12 is referred to as a partial cylinder because they do not form a closed cylinder but instead have a free, unconnected edge 14 along the length of each cylinder. In other words, for an embodiment in which the partial cylinder is a circular cylinder, a cross section of each partial cylinder may be an arc of a circle in the case that the edge 14 does not overlap, and not a complete circle. In the case that the edge 14 overlaps, the cross-section may be have a spiral shape, or a portion that is spiraled, such that the edge 14 overlaps to the inside of the other side of the barrel 12 (see e.g., the embodiments of FIGS. 5 and 7).

In alternative embodiments, the barrels 12 may have a partial polygonal cross-section, such as a pentagon, hexagon, heptagon, octagon, etc, or other partially closed shape consisting of line and/or curve segments. The shape is partially closed because it is not connected along the free, unconnected edge of the barrel. As with the circular cylinder shapes, the edge 14 may overlap or not overlap.

For ease in the illustration, the clot removal device 10 is shown in FIG. 1 as a single sheet of material, such as plate 16 to better appreciate a perspective view the barrels 12a and 12b. In this embodiment, the coupling plate 16 is configured to be connected to a wire or other actuating mechanism for manipulating the clot removal device 10, including advancing and withdrawing the clot removal device 10 within a blood vessel. The coupling plate 16 has a connector 18. In the embodiment of FIGS. 1 and 2, the connector 18 comprises an aperture through the coupling plate 16. The connector 18 may be other suitable connectors such as a pin, clip, weld, adhesive, antenna lap joint (as shown in FIG. 3), etc.

The clot removal device 10 is formed from clot engaging structure 20, such as a single, flat sheet of material having interconnected struts forming an open cell pattern as shown in FIG. 3. The clot engaging structure 20, and thus the clot removal device 10, may be made of any suitable material, such as an appropriate metal or polymer which can be formed into the clot removal device 10. For example, the clot engaging structure 20 is composed of shape memory, self-expandable and biocompatible materials, such as Nitinol. The clot removal device 10 is preferably manufactured by laser cutting the shape memory material sheet of the clot engaging structure 20.

The clot engaging structure 20 comprises a plurality of interconnected struts 25 (e.g., undulating elements, wires or the like) forming an open cell 27 pattern. The struts 25 may include a plurality of longitudinal undulating elements with adjacent undulating elements being out-of-phase with one another and connected in a manner to form the plurality of diagonally disposed cells 27. The clot engaging structure 20 includes a radially constrained configuration and a radially expanded configuration. When the clot engaging structure 20 is in the radially expanded configuration, a first portion 26 of the clot engaging structure 20 is rolled about itself in a clockwise direction to form a first barrel 12a, and a second portion 28 of the clot engaging structure 20 is rolled about itself in a counter-clockwise direction to form a second barrel 12b that extends in a side-by-side configuration with the first barrel 12a, as shown for example in FIGS. 1-2, 4-7 and 12-15.

The clot engaging structure 20 further comprises a barrel portion 22 which is formed into the barrels 12 (i.e., 12a, 12b), and an extension piece 24 extending from the barrel portion 22 which forms the coupling plate 16. The barrel portion 22 includes a first edge 26 which forms the free edge 14 of a first barrel 12a and a second edge 28 opposing the first edge 26 which forms the free edge 14 of the other barrel 12b. The clot engaging structure 20 is formed into the clot removal device 10 by forming the barrel portion 22 into the barrels 12a-b using a tool such as a mandrel. For instance, the first edge 26 of the sheet is rolled over the tool to form the first barrel 12a having a longitudinal axis parallel to the first edge 26. The first edge 26 is positioned by the forming such that it is left free and unconnected from the remainder of the first barrel 12a along the length of the first barrel 12a. Then, the second edge 28 is rolled over a tool to form the second barrel 12b adjacent and substantially parallel to the first barrel 12a. The second edge 28 is left free and unconnected from the remainder of the second barrel 12b along the length of the second barrel 12b.

In the embodiment of FIGS. 1-2 and 4-7, the first edge 26 and second edge 28 are rolled in opposite orientations relative to the middle of the sheet, thereby forming a “FIG. 8”-like cross-sectional shape, wherein the first edge 26 is rolled in a clockwise direction (e.g., downwardly oriented) and the second edge 28 is rolled in a counter-clockwise direction (e.g., upwardly oriented), from the middle of clot engaging structure 20.

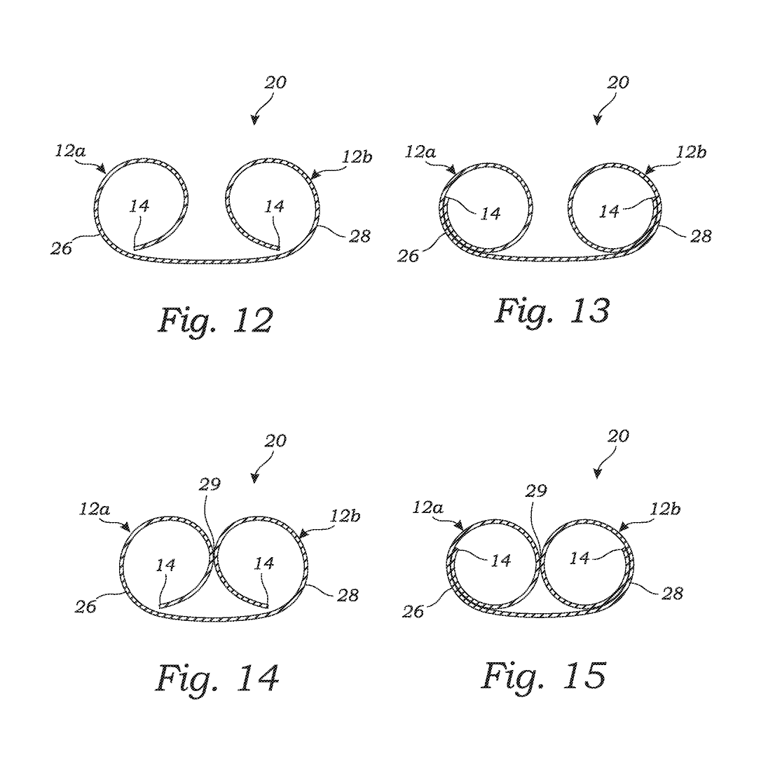

In the alternative embodiments depicted in FIGS. 12-15, the first barrel 12a and second barrel 12b are rolled in the same orientation relative to or towards the middle of the sheet, wherein the first edge 26 is rolled in a clockwise direction and the second edge 28 is rolled in a counter-clockwise direction, with both edges 26 and 28 being upwardly oriented towards the middle of clot engaging structure 20.

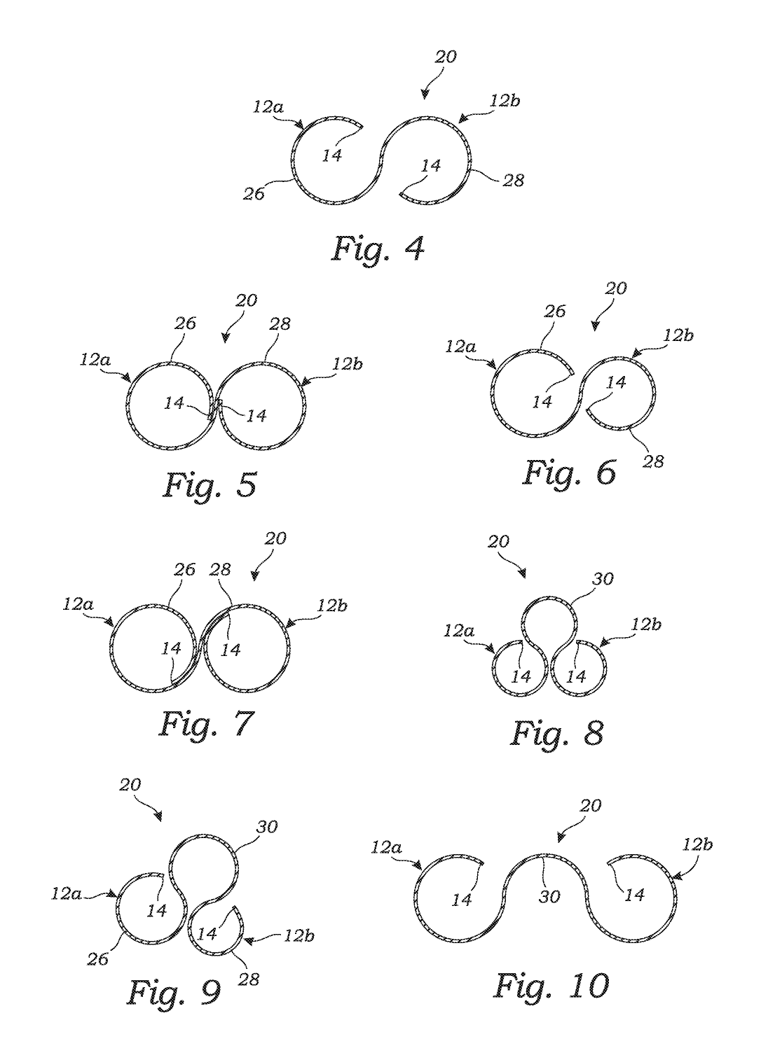

Turning now to FIGS. 4-10, the cross-section of the respective barrels 12 for several alternative embodiments of the clot engaging structure 20 are shown. Each of the alternative embodiments may have all of the features as described above for the clot removal device 10 of FIGS. 1-3, including being formed from a single sheet of material and/or the sheet of material comprising a plurality of struts 25 and cells 27 structures, as shown in FIG. 3.

FIG. 4 shows a cross-section of the clot engaging structure 20 having two barrels 12 in which the free edge 14 of each of the barrels 12 is further from the opposing side of the respective barrel 12 such that the free edge 14 is spaced further from the respective barrel 12. As shown in FIG. 4, the first portion 26 terminates along the first edge 14 that does not overlap with a remainder of the first barrel 12a, and the second portion 28 terminates along a second edge 14 that does not overlap with a remainder of the second barrel 12b, when the clot engaging structure 20 is in the radially expanded configuration. This provides a larger opening between the free edges 14 (e.g., less material interaction) and the remainder of the barrel 12 which may make the barrels 12 more compressible because the free edges 14 can be moved a larger distance before contacting the respective other side of the barrels 12.

FIG. 5 shows a cross-section of the clot engaging structure 20 having two barrels 12 in which the free edges 14 of each of the barrels 12 overlaps the other side of the barrel 12. In this case, the barrels 12 have a spiral shape, or at least a portion that is spiral shaped, such that the free edge 14 overlaps to the inside of opposing side of the barrel 12. As shown in FIG. 5, the first portion 26 terminates along the first edge 14 that overlaps with at least a portion of the first barrel 12a, and second portion 28 terminates along the second edge 14 that overlaps with at least a portion of the second barrel 12b when the clot engaging structure 20 is in the radially expanded configuration.

It should be appreciated that, in any of the embodiments described herein, the barrels 12 may have all or some barrels 12 having free edges 14 that overlap with portions of the barrels, as shown in FIGS. 5, 7, 12 and 15, or all or some barrels 12 having free edges 14 that do not overlap with portions of the barrels, as shown in FIGS. 2, 4, 6, 8-10, 12 and 15, when the clot engaging structure 20 is in the radially expanded configuration.

FIG. 6 shows a cross-section of the clot engaging structure 20 having two barrels 12 in which the diameter of each barrel 12 is different. In the embodiment of FIG. 6, the barrel 12a has a larger diameter than the barrel 12b or the barrel 12b has a smaller diameter than the barrel 12a, when the clot engaging structure 20 is in the radially expanded configuration. Accordingly, in any of the embodiments described herein, the barrels 12 may have the same or similar size and shape, or the barrels 12 may have different sizes and shapes.

FIG. 7 shows a cross-section having two barrels 12 similar to FIG. 5, except that the free edge 14 of each barrel overlaps more than the free edges 14 in the embodiment of FIG. 5.

FIG. 8 shows a cross-section of the clot engaging structure 20 having two barrels 12 and including an intermediate portion 30 disposed between the two barrels 12. The intermediate portion 30 includes a loop and/or a third barrel when the clot engaging structure 20 is in the radially expanded configuration.

Each of the barrels 12 and 30 are the same or similar size in the embodiment of FIG. 8.

FIG. 9 shows a cross-section of the clot engaging structure 20 having two barrels 12 and the intermediate portion 30, similar to the embodiment of FIG. 8, except that the two barrels and the intermediate portion 30 each have a different size.

FIG. 10 shows a cross-section of the clot engaging structure 20 having the barrels 12 and the intermediate portion 30 similar to the embodiment of FIG. 8, except that the intermediate portion 30 has a larger opening between the edges 14 of the respective barrels 12a and 12b.

The operation of each of the embodiments of the clot removal device 10 is basically the same. The clot removal device 10 is attached to a wire or actuating mechanism by connecting the wire or actuating device to the connector 18. The clot removal device is inserted into an insertion catheter by compressing the barrels 12 of the clot removal device 10. The insertion catheter and clot removal device are inserted into the vasculature of the patient and advanced through the vasculature to position the clot removal device 10 just distal to the clot. The clot removal device 10 is removed from the intravascular catheter. This may be done by advancing the clot removal device 10 relative to the intravascular catheter, or by withdrawing the intravascular catheter relative to the clot removal device 10, or by a combination of both (e.g., advancing the clot removal and withdrawing the catheter). The barrels 12 of the clot removal device 10 then expand within the blood vessel into the clot. The clot engaging structure 20 may be biased to expand, or is otherwise expandable from, the radially constrained configuration to the radially expanded configuration when deployed from a delivery catheter into a blood vessel. The clot removal device 10 is then moved proximally to ensnare the clot by pulling the wire or other actuating mechanism connected to the connector 18. The clot removal device 10 is then withdrawn proximally to remove the clot from the blood vessel. For instance, the clot removal device 10 and clot may be withdrawn into the insertion catheter, and then the entire assembly including the clot removal device 10 and catheter are withdrawn from the vasculature.

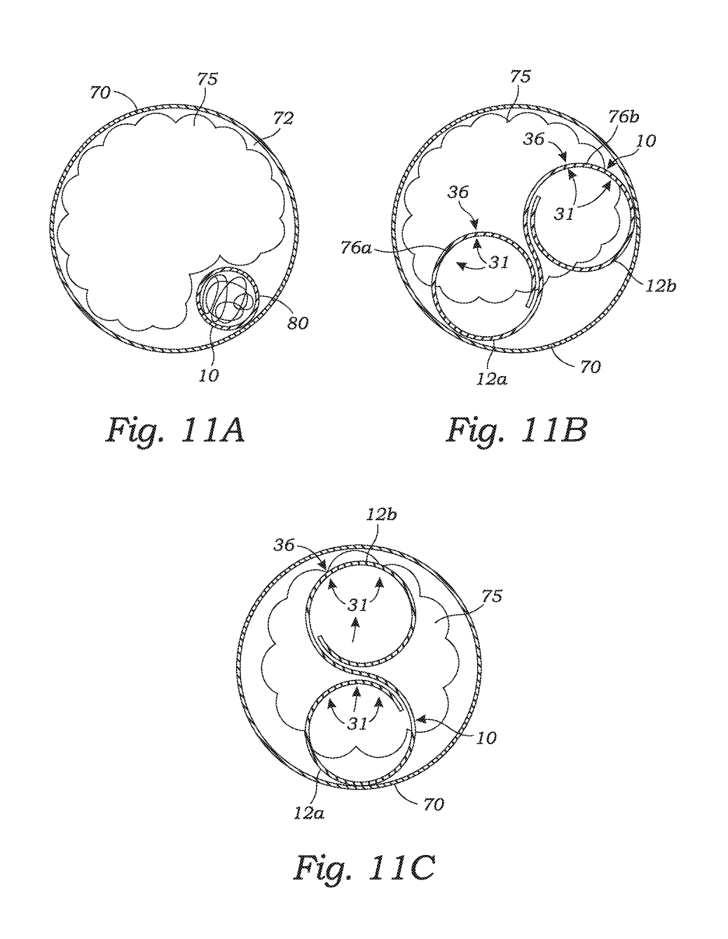

FIGS. 11A-C illustrate exemplary use of the clot removal device 10 according to the disclosed inventions. The clot removal device 10 disposed in a target site of a patient's blood vessel 70 (e.g., within a lumen 72 and adjacently located to a clot 75); the device 10 is radially constrained by a catheter 80, as shown in FIG. 11A. The clot removal device 10 is either pushed distally relative to a catheter 80, or the catheter 80 is withdrawn proximally relative to the clot removal device 10 (or some of each) (not shown), in order to deploy the clot removal device 10 out of the catheter 80 and into the lumen 72 of the blood vessel 70, and allow the no-longer radially constrained clot removal device 10 to radially expand within the blood vessel 70 in order to engage, ensnare and encapsulate the clot 75. When the clot removal device 10 is deployed, the barrels 12 of device 10 expand (decompress), and unroll due to radial expansion forces.

The clot removal device 10, in particular, the barrels 12 may assume a variety of orientations relative to the clot 75 when deployed within the patient's blood vessel 70, such as for example the orientations of FIGS. 11 B-C or any range therebetween. For example, the device 10 may assume an orientation having barrels 12a and 12b in contact with the blood vessel 70 when deployed, so that the barrels 12a-b assist the device 10 to overcome resistive forces 36 of the clot 75, which in turn allows the device to penetrate the clot 75 at least at two points of entry 76a-b, in order to engage, ensnare and/or capture the clot 75, as shown in FIG. 11B. In another example, the device 10 may assume an orientation having one of the barrels 12a in contact with the blood vessel 70 when deployed, the barrel 12a will further assist the device 10 to overcome resistive forces 36 of the clot 75 by pushing the barrel 12b further into the clot 75 engaging, ensnaring and/or capturing the clot 75, as shown in FIG. 11C.

In yet another example, the device 10 may assume any orientation in a range between the orientations of FIGS. 11B-C, which in turns may provide a combination of the advantages of said orientations of FIGS. 11B-C to the device 10 (e.g., multiple points of entry to the clot and/or assistance to overcome the resistive forces of the clot).

FIG. 12 shows a cross-section of another embodiment of the clot engaging structure 20, in which the first portion 26 terminates along the first edge 14 that does not overlap with a remainder of the first barrel 12a, and the second portion 28 terminates along a second edge 14 that does not overlap with a remainder of the second barrel 12b, respectively, when the clot engaging structure 20 is in the radially expanded configuration. This embodiment provides a larger opening between the free edges 14 (e.g., less material interaction) and the remainder of the barrels 12, which may make the barrels 12 more compressible because the free edges 14 can be moved a larger distance before contacting the respective other side of the barrels 12. In this embodiment, the clot engaging structure 20 includes a space or separation between barrels 12a and 12b (e.g., barrels 12 are not in contact with each other), when the clot engaging structure 20 is in the radially expanded configuration.

FIG. 13 shows a cross-section of yet another embodiment of the clot engaging structure 20, in which the first portion 26 terminates along the first edge 14 that overlaps with at least a portion of the first barrel 12a, and second portion 28 terminates along the second edge 14 that overlaps with at least a portion of the second barrel 12b, respectively, when the clot engaging structure 20 is in the radially expanded configuration. In this embodiment, the clot engaging structure 20 includes a space or separation between barrels 12a and 12b (e.g., barrels 12 are not in contact with each other), when the clot engaging structure 20 is in the radially expanded configuration.

FIG. 14 shows a cross-section of still another embodiment of the clot engaging structure 20, in which the first portion 26 terminates along the first edge 14 that does not overlap with a remainder of the first barrel 12a, and the second portion 28 terminates along a second edge 14 that does not overlap with a remainder of the second barrel 12b, respectively, when the clot engaging structure 20 is in the radially expanded configuration. In this embodiment, the first portion 26 of the barrel 12a is in contact with at least one point or section 29 of the second portion 28 of the second barrel 12b, when the clot engaging structure 20 is in the radially expanded configuration.

FIG. 15 shows a cross-section of yet another embodiment of the clot engaging structure 20, in which the first portion 26 terminates along the first edge 14 that overlaps with at least a portion of the first barrel 12a, and second portion 28 terminates along the second edge 14 that overlaps with at least a portion of the second barrel 12b, respectively, when the clot engaging structure 20 is in the radially expanded configuration. In this embodiment, the first portion 26 of the barrel 12a is in contact with at least one point or section 29 of the second portion 28 of the second barrel 12b, when the clot engaging structure 20 is in the radially expanded configuration.

It will be appreciated that the use of the various clot removal devices 10 shown in FIGS. 1-15 may also be used in other suitable medical applications. Furthermore, it should be apparent to those of ordinary skill in the art that many modifications may be made to the illustrated and herein described embodiments may be made without departing from the scope of the appended claims, and that the proper scope of the disclosed inventions should be limited only as defined in the appended claims, and their full legal equivalents, so as to encompass all such modifications and equivalents.

What is claimed is:

1. A clot removal device, comprising:
a clot engaging structure comprising a plurality of interconnected struts forming an open cell pattern, the clot engaging structure having a radially constrained configuration and a radially expanded configuration,
wherein when the clot engaging structure is in the radially expanded configuration, a first portion of the clot engaging structure is rolled about itself in a clockwise direction to form a first barrel, and a second portion of the clot engaging structure is rolled about itself in a counter-clockwise direction to form a second barrel that extends in a side-by-side configuration with the first barrel.

2. The clot removal device of claim 1, wherein the first portion terminates along a first edge that does not overlap with a remainder of the first barrel when the clot engaging structure is in the radially expanded configuration.

3. The clot removal device of claim 1, wherein the second portion terminates along a second edge that does not overlap with a remainder of the second barrel when the clot engaging structure is in the radially expanded configuration.

4. The clot removal device of claim 1, wherein the first portion terminates along a first edge that overlaps with at least a portion of the first barrel when the clot engaging structure is in the radially expanded configuration.

5. The clot removal device of claim 4, wherein the second portion terminates along a second edge that overlaps with at least a portion of the second barrel when the clot engaging structure is in the radially expanded configuration.

6. The clot removal device of 5, wherein when the clot engaging structure is in the radially expanded configuration, the first barrel has a first barrel diameter, and the second barrel has a second barrel diameter that is smaller than the first barrel diameter.

7. The clot removal device of claim 1, the clot engaging structure further comprising an intermediate portion disposed between the first and second portions.

8. The clot removal device of claim 7, wherein the intermediate portion comprises a loop and/or a third barrel when the clot engaging structure is in the radially expanded configuration.

9. The clot removal device of claim 1, wherein the clot engaging structure is biased to expand, or is otherwise expandable from, the radially constrained configuration to the radially expanded configuration when deployed from a delivery catheter into a blood vessel.

10. The clot removal device of claim 1, wherein when the clot engaging structure is delivered to a targeted vascular site proximate a vascular obstruction, and moved or allowed to move from the radially constrained configuration to the radially expanded configuration, the first and second barrels move, be moved, or be allowed to move, respectively, from a radially constrained configuration to a radially expanded configuration to thereby ensnare or encapsulate the vascular obstruction or portions thereof between or within the first and second barrels.
